(19) United States
(12) Patent Application Publication
(10) Pub. No.: US 2003/0129242 A1
(43) Pub. Date: Jul. 10, 2003

(54) Sterile filtered nanoparticulate formulations of budesonide and beclomethasone having tyloxapol as a surface stabilizer

(76) Inventors: H. William Bosch, Bryn Mawr, PA (US); Donna M. Marcera, Limerick, PA (US); Kevin D. Ostrander, Reading, PA (US); Niels P. Ryde, Malvern, PA (US); Douglas A. White, King of Prussia, PA (US)

Correspondence Address: Michele M. Simkin, FOLEY & LARDNER, Washington Harbour, 3000 K Street, N.W., Suite 500, Washington, DC, 20007-5143, US

(21) Appl. No.: 10/035,324

(22) Filed: Jan. 4, 2002

(51) Int. Cl. A61K 31/573; A61L 9/04; A61K 9/14

(52) U.S. Cl. 424/489; 514/179; 424/46

(57) ABSTRACT

The invention relates to sterile filtered nanoparticulate compositions of beclomethasone and/or budesonide having as a surface stabilizer tyloxapol and, optionally, one or more secondary surface stabilizers adsorbed onto the surfaces thereof. The nanoparticulate compositions have an optimal effective average particle size of less than about 150 nm.

### FIELD OF THE INVENTION

This invention is directed to nanoparticulate compositions of beclomethasone and/or budesonide having tyloxapol as a surface stabilizer, and to methods for the preparation and use of such compositions. The formulations are sterile filtered and are thus useful in pharmaceutical compositions.

### BACKGROUND OF THE INVENTION

A. Background Regarding Nanoparticulate Compositions

Nanoparticulate compositions, first described in U.S. Pat. No. 5,145,684 (“the '684 patent”), are particles consisting of a poorly soluble active agent having adsorbed onto the surface thereof a non-crosslinked surface stabilizer. The '684 patent also describes methods of making such nanoparticulate compositions. Nanoparticulate compositions are desirable because with a decrease in particle size, and a consequent increase in surface area, a composition is rapidly dissolved and absorbed following administration. Methods of making such compositions are described, for example, in U.S. Pat. Nos. 5,518,187 and 5,862,999, both for “Method of Grinding Pharmaceutical Substances,” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. Nos. 5,298,262 for “Use of Ionic Cloud Point Modifiers to Prevent Particle Aggregation During Sterilization;” U.S. Pat. No. 5,302,401 for “Method to Reduce Particle Size Growth During Lyophilization;” U.S. Pat. No. 5,318,767 for “X-Ray Contrast Compositions Useful in Medical Imaging;” U.S. Pat. No. 5,326,552 for “Novel Formulation For Nanoparticulate X-Ray Blood Pool Contrast Agents Using High Molecular Weight Non-ionic Surfactants;” U.S. Pat. No. 5,328,404 for “Method of X-Ray Imaging Using Iodinated Aromatic Propanedioates;” U.S. Pat. No. 5,336,507 for “Use of Charged Phospholipids to Reduce Nanoparticle Aggregation;” U.S. Pat. No. 5,340,564 for “Formulations Comprising Olin 10-G to Prevent Particle Aggregation and Increase Stability;” U.S. Pat. No. 5,346,702 for “Use of Non-Ionic Cloud Point Modifiers to Minimize Nanoparticulate Aggregation During Sterilization;” U.S. Pat. No. 5,349,957 for “Preparation and Magnetic Properties of Very Small Magnetic-Dextran Particles;” U.S. Pat. No. 5,352,459 for “Use of Purified Surface Modifiers to Prevent Particle Aggregation During Sterilization;” U.S. Pat. Nos. 5,399,363 and 5,494,683, both for “Surface Modified Anticancer Nanoparticles;” U.S. Pat. No. 5,401,492 for “Water Insoluble Non-Magnetic Manganese Particles as Magnetic Resonance Enhancement Agents;” U.S. Pat. No. 5,429,824 for “Use of Tyloxapol as a Nanoparticulate Stabilizer;” U.S. Pat. No. 5,447,710 for “Method for Making Nanoparticulate X-Ray Blood Pool Contrast Agents Using High Molecular Weight Non-ionic Surfactants;” U.S. Pat. No. 5,451,393 for “X-Ray Contrast Compositions Useful in Medical Imaging;” U.S. Pat. No. 5,466,440 for “Formulations of Oral Gastrointestinal Diagnostic X-Ray Contrast Agents in Combination with Pharmaceutically Acceptable Clays;” U.S. Pat. No. 5,470,583 for “Method of Preparing Nanoparticle Compositions Containing Charged Phospholipids to Reduce Aggregation;” U.S. Pat. No. 5,472,683 for “Nanoparticulate Diagnostic Mixed Carbamic Anhydrides as X-Ray Contrast Agents for Blood Pool and Lymphatic System Imaging;” U.S. Pat. No. 5,500,204 for “Nanoparticulate Diagnostic Dimers as X-Ray Contrast Agents for Blood Pool and Lymphatic System Imaging;” U.S. Pat. No. 5,518,187 for “Method of Grinding Pharmaceutical Substances;” U.S. Pat. No. 5,518,738 for “Nanoparticulate NSAID Formulations;” U.S. Pat. No. 5,521,218 for “Nanoparticulate Iododipamide Derivatives for Use as X-Ray Contrast Agents;” U.S. Pat. No. 5,525,328 for “Nanoparticulate Diagnostic Diatrizoxy Ester X-Ray Contrast Agents for Blood Pool and Lymphatic System Imaging;” U.S. Pat. No. 5,543,133 for “Process of Preparing X-Ray Contrast Compositions Containing Nanoparticles;” U.S. Pat. No. 5,552,160 for “Surface Modified NSAID Nanoparticles;” U.S. Pat. No. 5,560,931 for “Formulations of Compounds as Nanoparticulate Dispersions in Digestible Oils or Fatty Acids;” U.S. Pat. No. 5,565,188 for “Polyalkylene Block Copolymers as Surface Modifiers for Nanoparticles;” U.S. Pat. No. 5,569,448 for “Sulfated Non-ionic Block Copolymer Surfactant as Stabilizer Coatings for Nanoparticle Compositions;” U.S. Pat. No. 5,571,536 for “Formulations of Compounds as Nanoparticulate Dispersions in Digestible Oils or Fatty Acids;” U.S. Pat. No. 5,573,749 for “Nanoparticulate Diagnostic Mixed Carboxylic Anydrides as X-Ray Contrast Agents for Blood Pool and Lymphatic System Imaging;” U.S. Pat. No. 5,573,750 for “Diagnostic Imaging X-Ray Contrast Agents;” U.S. Pat. No. 5,573,783 for “Redispersible Nanoparticulate Film Matrices With Protective Overcoats;” U.S. Pat. No. 5,580,579 for “Site-specific Adhesion Within the GI Tract Using Nanoparticles Stabilized by High Molecular Weight, Linear Poly(ethylene Oxide) Polymers;” U.S. Pat. No. 5,585,108 for “Formulations of Oral Gastrointestinal Therapeutic Agents in Combination with Pharmaceutically Acceptable Clays;” U.S. Pat. No. 5,587,143 for “Butylene Oxide-Ethylene Oxide Block Copolymers Surfactants as Stabilizer Coatings for Nanoparticulate Compositions;” U.S. Pat. No. 5,591,456 for “Milled Naproxen with Hydropropyl Cellulose as Dispersion Stabilizer;” U.S. Pat. No. 5,593,657 for “Novel Barium Salt Formulations Stabilized by Non-ionic and Anionic Stabilizers;” U.S. Pat. No. 5,622,938 for “Sugar Based Surfactant for Nanocrystals;” U.S. Pat. No. 5,628,981 for “Improved Formulations of Oral Gastrointestinal Diagnostic X-Ray Contrast Agents and Oral Gastrointestinal Therapeutic Agents;” U.S. Pat. No. 5,643,552 for “Nanoparticulate Diagnostic Mixed Carbonic Anhydrides as X-Ray Contrast Agents for Blood Pool and Lymphatic System Imaging;” U.S. Pat. No. 5,718,388 for “Continuous Method of Grinding Pharmaceutical Substances;” U.S. Pat. No. 5,718,919 for “Nanoparticles Containing the R(−)Enantiomer of Ibuprofen;” U.S. Pat. No. 5,747,001 for “Aerosols Containing Beclomethasone Nanoparticle Dispersions;” U.S. Pat. No. 5,834,025 for “Reduction of Intravenously Administered Nanoparticulate Formulation Induced Adverse Physiological Reactions;” U.S. Pat. No. 6,045,829 “Nanocrystalline Formulations of Human Immunodeficiency Virus (HIV) Protease Inhibitors Using Cellulosic Surface Stabilizers;” U.S. Pat. No. 6,068,858 for “Methods of Making Nanocrystalline Formulations of Human Immunodeficiency Virus (HIV) Protease Inhibitors Using Cellulosic Surface Stabilizers;” U.S. Pat. No. 6,153,225 for “Injectable Formulations of Nanoparticulate Naproxen;” U.S. Pat. No. 6,165,506 for “New Solid Dose Form of Nanoparticulate Naproxen;” U.S. Pat. No. 6,221,400 for “Methods of Treating Mammals Using Nanocrystalline Formulations of Human Immunodeficiency Virus (HIV) Protease Inhibitors;” U.S. Pat. No. 6,264,922 for “Nebulized Aerosols Containing Nanoparticle Dispersions;” U.S. Pat. No. 6,267,989 for “Methods for Preventing Crystal Growth and Particle Aggregation in Nanoparticle Compositions;” and U.S. Pat. No. 6,270,806 for “Use of PEG-Derivatized Lipids as Surface Stabilizers for Nanoparticulate Compositions,” all of which are specifically incorporated by reference.

Amorphous small particle compositions are described in, for example, U.S. Pat. Nos. 4,783,484 for “Particulate Composition and Use Thereof as Antimicrobial Agent,” U.S. Pat. No. 4,826,689 for “Method for Making Uniformly Sized Particles from Water-Insoluble Organic Compounds,” U.S. Pat. No. 4,997,454 for “Method for Making Uniformly-Sized Particles From Insoluble Compounds,” U.S. Pat. No. 5,741,522 for “Ultrasmall, Non-aggregated Porous Particles of Uniform Size for Entrapping Gas Bubbles Within and Methods,” and U.S. Pat. No. 5,776,496, for “Ultrasmall Porous Particles for Enhancing Ultrasound Back Scatter.

B. Background Relating to Sterilization of Nanoparticulate Compositions

There are two generally accepted methods for sterilizing pharmaceutical products: heat sterilization and sterile filtration.

1. Heat Sterilization of Nanoparticulate Compositions

One of the problems that may be encountered with heat sterilization of nanoparticulate compositions is the solubilization and subsequent recrystallization of the component drug particles. This process results in an increase in the size distribution of the drug particles. In addition, some nanoparticulate formulations also exhibit particle aggregation following exposure to elevated temperatures for heat sterilization.

Crystal growth and particle aggregation in nanoparticulate preparations are highly undesirable for several reasons. The presence of large crystals in the nanoparticulate composition may cause undesirable side effects, especially when the preparation is in an injectable formulation. This is also true for particle aggregation, as injectable formulations preferably have an effective average particle size of no greater than 250 nm. Larger particles formed by particle aggregation and recrystallization can interfere with blood flow, causing pulmonary embolism and death.

In addition, with both injectable and oral formulations the presence of large crystals, and therefore varying particle sizes, and/or particle aggregation can change the pharmacokinetic profile of the administered drug. For oral formulations, the presence of large crystals or aggregates creates a variable bioavailability profile because smaller particles dissolve faster than the larger aggregates or larger crystal particles. A faster rate of dissolution is associated with greater bioavailability and a slower rate of dissolution is associated with a lower bioavailability. This is because bioavailability is proportional to the surface area of an administered drug and, therefore, bioavailability increases with a reduction in the particle size of the dispersed agent (see U.S. Pat. No. 5,662,833). With a composition having widely varying particle sizes, bioavailability becomes highly variable and inconsistent and dosage determinations become difficult. Moreover, because such crystal growth and particle aggregation are uncontrollable and unpredictable, the quality of the nanoparticulate compositions is inconsistent. For intravenously injected particulate formulations, the presence of large crystals or aggregates can induce an immune systems response which causes the larger particles to be transported by macrophage cells to the liver or spleen and metabolized, in addition to the embolytic effects described above.

Aggregation of nanoparticle compositions upon heating is directly related to the precipitation of the surface stabilizer at temperatures above the cloud point of the surface stabilizer. At this point, the bound surface stabilizer molecules are likely to dissociate from the nanoparticles and precipitate, leaving the nanoparticles unprotected. The unprotected nanoparticles then aggregate into clusters of particles.

Several methods have been suggested in the prior art for preventing such crystal growth and particle aggregation following heat sterilization, including adding a cloud point modifier or crystal growth modifier to the nanoparticulate composition and purifying the surface stabilizer. For example, U.S. Pat. No. 5,298,262 describes the use of an anionic or cationic cloud point modifier in nanoparticulate compositions and U.S. Pat. No. 5,346,702 describes nanoparticulate compositions having a nonionic surface stabilizer and a non-ionic cloud point modifier. The cloud point modifier enables heat sterilization of the nanoparticulate compositions with low resultant particle aggregation. U.S. Pat. No. 5,470,583 describes nanoparticulate compositions having a non-ionic surface stabilizer and a charged phospholipid as a cloud point modifier.

The prior art also describes methods of limiting crystal growth in a nanoparticulate composition by adding a crystal growth modifier (see U.S. Pat. Nos. 5,662,883 and 5,665,331). In addition, U.S. Pat. No. 5,302,401 describes nanoparticulate compositions having polyvinylpyrrolidone (PVP) as a surface stabilizer and sucrose as a cryoprotectant (allowing the nanoparticles to be lyophilized). The compositions exhibit minimal particle aggregation following lyophilization.

All of these various prior art methods share one common feature: they require an additional substance added to the nanoparticulate formulation to inhibit or prevent crystal growth and particle aggregation of the nanoparticulate composition. The addition of such a substance can be detrimental as it may induce adverse effects, particularly for injectable formulations. Thus, this minimizes the usefulness of such substances in pharmaceutical compositions. In addition, the requirement of an additional substance to obtain a stable composition increases production costs.

Another method of limiting particle aggregation or crystal growth of nanoparticulate compositions during sterilization known prior to the present invention was the use of purified surface stabilizers. U.S. Pat. No. 5,352,459 describes nanoparticulate compositions having a purified surface stabilizer (having less than 15% impurities) and a cloud point modifier. Purification of surface stabilizers can be expensive and time consuming, thus significantly raising production costs of compositions requiring such stabilizers to produce a stable nanoparticulate composition.

2. Sterile Filtration

Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize conventional suspensions of micron-sized drug particles because the drug substance particles are too large to pass through the membrane pores. In principle, 0.2 μm filtration can be used to sterilize nanoparticulate compositions. However, because nanoparticulate compositions have a size range, many of the particles of a typical nanoparticulate composition having an average particle size of 200 nm may have a size greater than 200 nm. Such larger particles tend to clog the sterile filter. Thus, only nanoparticulate compositions having very small average particle sizes can be sterile filtered.

C. Background Relating to Beclomethasone and Budesonide

Budesonide and beclomethasone are anti-inflammatory glucocorticoids useful in the treatment of diseases such as asthma. See William E. Serafin, “Therapeutic compounds Used in the Treatment of Asthma”, Goodman and Gilman's: The Pharmacological Basis of Therapeutics, Ninth Edition 659-682 (J. G. Hardman et al., eds., McGraw Hill 1996). The prior art discloses the preparation of aerosol formulations of nanoparticulate beclomethasone dipropionate in U.S. Pat. No. 5,747,001.

Beclomethasone dipropionate has the following structural formula:

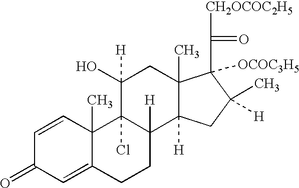

It is a white powder with a molecular weight of 521.25 and is very slightly soluble in water.

Budesonide has the following formula:

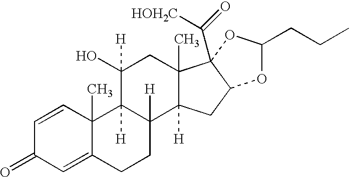

Budesonide is designated chemically as (RS)-11,16, 17,21-Tetrahydroxy-pregna-1,4-diene-3,20-dione cyclic 16,17-acetal with butraldehyde. Budesonide is provided as the mixture of two epimers (22R and 22S). The empirical formula of budesonide is C₂₅H₃₄O₆ and its molecular weight is 430.5.

Budesonide is a white to off-white odorless powder that is practically insoluble in water and in heptane, sparingly soluble in ethanol, and freely soluble in chloroform.

Glucocorticosteroids have been shown to have a wide range of inhibitory activities against multiple cell types (e.g., mast cells, eosinophils, neutrophils, macrophages, and lymphocytes) and mediators (e.g., histamine, eicosanoids, leukotrienes and cytokines) involved in allergic and nonallergic/irritant-mediated inflammation. Corticoids affect the delayed (6 hour) response to an allergen challenge more than the histamine-associated immediate response (20 minutes).

D. Inhalation Treatment with Glucocorticoids

Administration by inhalation of glucocorticoids, compared with oral administration, reduces the risk of systemic side effects. The reduced risk of side effect arises from the mode of administration because glucocorticoids are highly active topically and only weakly active systemically, thereby minimizing effects on the pituitary-adrenal axis, the skin, and the eye. Side effects associated with inhalation therapy are primarily oropharyngeal candidiasis and dysphonia (due to atrophy of laryngeal muscles). Oral glucocorticoids cause atrophy of the dermis with thin skin, striae, and ecchymoses but inhaled glucocorticoids do not cause similar changes in the respiratory tract.

Other advantages of inhaled over oral administration include direct deposition of steroid in the airways which generally provides more predictable administration. The oral doses required for adequate control vary substantially, whereas inhaled glucocorticoids are usually effective within a narrower range. There are, however, a number of factors that influence the availability of inhaled glucocorticoids: extent of airway inflammation; degree of lung metabolism; amount of drug swallowed and metabolized in the GI tract; the patient's ability to coordinate the release and inspiration of the medication; type of glucocorticoid; and the delivery system.

However, the U.S. Food and Drug Administration has recently issued guidelines requiring inhaled products to be sterile. This is problematic for aerosol formulations of nanoparticulate drugs, as heat sterilization can result in crystal growth and particle aggregation, and sterile filtration can be difficult because of the required small particle size of the composition.

There is a need in the art for sterile dosage forms of nanoparticulate beclomethasone and budesonide. The present invention satisfies this need.

### SUMMARY OF THE INVENTION

The present invention is directed to the unexpected discovery that nanoparticulate compositions of beclomethasone or budesonide having tyloxapol as a surface stabilizer can be readily sterilized by sterile filtration.

The compositions of the invention comprise nanoparticulate beclomethasone, budesonide, or a combination thereof, both having tyloxapol as a surface stabilizer. The compositions may also include one or more secondary surface stabilizers adsorbed onto the surface of the drugs.

The nanoparticulate compositions have an optimal effective average particle size of less than about 150 nm, less than about 120 nm, less than about 100 nm, less than about 80 nm, or less than about 50 nm. Because the compositions have such a small effective average particle size, they can be readily sterile filtered.

Another aspect of the present invention is directed to a method of making the nanoparticulate compositions of the invention. Such a method comprises contacting beclomethasone and/or budesonide with tyloxapol, and if desired one or more secondary surface stabilizers, for a time and under conditions sufficient to obtain a nanoparticulate composition having the desired particle size. The compositions can then be sterile filtered.

Yet another aspect of the invention is directed to a pharmaceutical composition comprising a sterile filtered nanoparticulate composition of the invention. The pharmaceutical composition comprises a therapeutically effective amount of a nanoparticulate composition of the invention in admixture with a pharmaceutically acceptable carrier.

Still another aspect of the present invention is directed to a method of treating a mammal suffering from a condition for which beclomethasone or budesonide is indicated, comprising administering to the mammal a therapeutically effective amount of a pharmaceutical composition of the present invention.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE PREFERRED EMBODIMENTS

The present invention is directed to nanoparticulate compositions of beclomethasone and/or budesonide having tyloxapol as a surface stabilizer, and optionally one or more secondary surface stabilizers. Surprisingly, the compositions have extremely small effective average particle sizes, which allow the compositions to be sterile filtered.

As taught in the '684 patent, not every combination of surface stabilizer and drug will result in a stable nanoparticulate composition. The discovery of the present invention is surprising as other surface stabilizers were found to be ineffective in attempts to make nanoparticulate compositions of beclomethasone and budesonide. Such stabilizers include hydroxypropyl methylcellulose, methyl cellulose, Pluronic F108®, polysorbates 20 and 80, and polyvinylpyrrolidine.

Even more surprising is that even when a nanoparticulate composition of budesonide and beclomethasone having one or more of the non-tyloxapol surface stabilizers was made, such a nanoparticulate composition could not successfully be sterile filtered.

Finally, it was surprisingly discovered that not all steroids having tyloxapol as a surface stabilizer can be reduced to a particle size small enough to be sterile filtered, as demonstrated by experiments with flunisolide and triamcinolone acetonide. Thus, the discovery of the present invention does not extend to a class of compounds; but rather is limited to the steroids budesonide and beclomethasone.

A. Nanoparticulate Compositions

The compositions of the invention comprise beclomethasone, budesonide, or a combination thereof as active agents, both compounds having tyloxapol adsorbed on the surface of the active agents as a surface stabilizer. One or more secondary surface stabilizers may also be adsorbed thereon. Such surface stabilizers physically adhere to the surface of the nanoparticulate active agent, but do not chemically react with the active agent or with each other. Individually adsorbed molecules of the surface stabilizer are essentially free of intermolecular crosslinkages.

As used herein, the term beclomethasone means free beclomethasone and its various mono- and diesters. Specifically included is beclomethazone dipropionate and its monohydrate. The term budesonide means free budesonide and its various mono- and diesters.

Budesonide may be given in a high inhaled dose with very low systemic effects, possibly because of its rapid metabolism. The high rapid systemic elimination of budesonide is due to extensive and rapid hepatic metabolism. Long term clinical studies have shown that inhaled budesonide is a pharmacologically safe drug. High doses of inhaled budesonide are highly effective and well tolerated when used in oral steroid replacement therapy. In addition, budesonide has exhibited benefits of long term control of asthma.

Beclomethasone and budesonide have a high affinity for intracellular glucocorticoid receptors but are rapidly metabolized to biologically inactive compounds. Asthma can usually be controlled with daily inhaled doses of beclomethasone or budesonide in the range of 200 to 800 micrograms. Doses up to 1000 microgram daily have little effect on pituitary-adrenal secretion in adults; larger doses may cause some (variable) dose-dependent suppression of secretion. Doses of 2000 microgram/day in adults have been associated with thinning of the skin, slight glucose intolerance, psychiatric disturbances (rarely), and cataracts (with long-term therapy). Beclomethasone in doses of 1000 to 2000 microgram/day (long term) has been associated with decreases in bone density.

The present invention also includes the nanoparticulate compositions of the invention formulated into pharmaceutical compositions together with one or more non-toxic physiologically acceptable carriers, adjuvants, or vehicles, collectively referred to as carriers, for parenteral injection, for oral administration in solid or liquid form, for rectal or topical administration, inhalable or nasal aerosol administration, and the like.

1. Surface Stabilizers

The nanoparticulate budesonide or beclomethasone has tyloxapol as a surface stabilizer adsorbed onto the surface of the drug particles. Tyloxapol is a (4-(1,1,3,3,-tetramethylbutyl)-phenol polymer with ethylene oxide and formaldehyde, which is a nonionic liquid polymer of the alkyl aryl polyether alcohol type, and is also known as superinone or triton. Tyloxapol is commercially available and/or can be prepared by techniques known in the art.

Tyloxapol is disclosed as being a useful nonionic surface active agent in a lung surfactant composition in U.S. Pat. No. 4,826,821 and as a stabilizing agent for 2-dimethylaminoethyl 4-n-butylaminobenzoate in U.S. Pat. No. 3,272,700. In addition, tyloxapol is taught as being a useful surface stabilizer for nanoparticulate compositions in U.S. Pat. No. 5,429,824.

In addition to tyloxapol as a surface stabilizer, optional secondary surface stabilizers are also contemplated. Useful secondary surface stabilizers include various polymers, low molecular weight oligomers, natural products, and surfactants. Preferred surface stabilizers include nonionic and ionic surfactants. Two or more secondary surface stabilizers may be employed in combination.

Representative examples of secondary surface stabilizers include cetyl pyridinium chloride, gelatin, casein, lecithin (phosphatides), dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as Tween 20® and Tween 80® (ICI Specialty Chemicals)); polyethylene glycols (e.g., Carbowaxs 3350® and 1450®, and Carbopol 934® (Union Carbide)), dodecyl trimethyl ammonium bromide, polyoxyethylene stearates, colloidal silicon dioxide, phosphates, sodium dodecylsulfate, carboxymethylcellulose calcium, hydroxypropyl celluloses (e.g., HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); a charged phospholipid such as dimyristoyl phophatidyl glycerol; dioctylsulfosuccinate (DOSS); Tetronic 1508® (T-1508) (BASF Wyandotte Corporation); dialkylesters of sodium sulfosuccinic acid (e.g., Aerosol OT®, which is a dioctyl ester of sodium sulfosuccinic acid (American Cyanamid)); Duponol P®, which is a sodium lauryl sulfate (D)uPont); Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-1OG® or Surfactant 10-G® (Olin Chemicals, Stamford, Conn.); Crodestas SL-40® (Croda, Inc.); SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; random copolymers of vinyl acetate and vinyl pyrrolidone, such as Plasdone® S630, and the like.

Particularly preferred secondary surface stabilizers are DOSS, sodium lauryl sulfate, hydroxypropylmethyl cellulose, benzalkonium chloride, and polyvinylpyrrolidine.

Most of these surface stabilizers are known pharmaceutical excipients and are described in detail in the Handbook of Phannaceutical Excipients, published jointly by the American Pharmaceutical Association and The Pharmaceutical Society of Great Britain (The Pharmaceutical Press, 1990), specifically incorporated herein by reference. The surface stabilizers are commercially available and/or can be prepared by techniques known in the art.

2. Nanoparticulate Beclomethasone or Budesonide/Surface Stabilizer Particle Size

The nanoparticulate compositions of the invention comprise nanoparticulate beclomethasone, budesonide, or a combination thereof, having an effective average particle less than about 150 nm, less than about 120 nm, less than about 100 nm, less than about 80 nm, or less than about 50 nm, as measured by conventional particle size measuring techniques well known to those skilled in the art. Such techniques include, for example, sedimentation field flow fractionation, photon correlation spectroscopy, light scattering, and disk centrifugation.

By “an effective average particle size less than about 150 nm” it is meant that at least 50% of the active agent particles have a weight average particle size of less than about 150 nm when measured by the above techniques. Preferably, at least 70%, 90%, or 95% of the active agent particles have an average particle size of less than about 150 nm.

3. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches. Examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quartemary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

4. Concentration of Nanoparticulate Beclomethasone or Budesonide and Tyloxapol

The relative amount of budesonide or beclomethasone and tyloxapol can vary widely. The optimal amount of drug and tyloxapol can depend, for example, upon the presence of secondary surface stabilizers, the particular intended dosage form, etc.

The concentration of tyloxapol can vary from about 0.01 to about 90%, from about 1 to about 75%, from about 10 to about 60%, or from about 10 to about 30% by weight, based on the total combined dry weight of the budesonide or beclomethasone and tyloxapol.

The concentration of the budesonide or beclomethasone can vary from about 99% to about 1%, from about 90% to about 10%, from about 80% to about 30%, or from about 80% to about 40% by weight, based on the total combined dry weight of the budesonide or beclomethasone and tyloxapol.

B. Methods of Making Nanoparticulate Formulations

The nanoparticulate beclomethasone or budesonide compositions of the invention can be made using, for example, milling, precipitation, or microfluidization techniques. Exemplary methods of making nanoparticulate compositions are described in the '684 patent. Methods of making nanoparticulate compositions are also described in U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” U.S. Pat. No. 5,665,331, for “Co-Microprecipitation of Nanoparticulate Pharmaceutical Agents with Crystal Growth Modifiers;” U.S. Pat. No. 5,662,883, for “Co-Microprecipitation of Nanoparticulate Pharmaceutical Agents with Crystal Growth Modifiers;” U.S. Pat. No. 5,560,932, for “Microprecipitation of Nanoparticulate Pharmaceutical Agents;” U.S. Pat. No. 5,543,133, for “Process of Preparing X-Ray Contrast Compositions Containing Nanoparticles;” U.S. Pat. No. 5,534,270, for “Method of Preparing Stable Drug Nanoparticles;” U.S. Pat. No. 5,510,118, for “Process of Preparing Therapeutic Compositions Containing Nanoparticles;” and U.S. Pat. No. 5,470,583, for “Method of Preparing Nanoparticle Compositions Containing Charged Phospholipids to Reduce Aggregation,” all of which are specifically incorporated by reference.

1. Milling to obtain Nanoparticulate Drug Dispersions

Milling of aqueous beclomethasone or budesonide to obtain a nanoparticulate dispersion comprises dispersing beclomethasone particles, budesonide particles, or a combination thereof in a liquid dispersion medium, followed by applying mechanical means in the presence of grinding media to reduce the particle size of the active agents to the desired effective average particle size.

The liquid dispersion medium can be any medium in which the active agent particles are poorly soluble. By “poorly soluble” it is meant that the drug has a solubility in the liquid dispersion medium of less than about 10 mg/ml, and preferably of less than about 1 mg/ml. A preferred liquid dispersion medium is water. However, the invention can also be practiced with other liquid media in which the drug is poorly soluble and dispersible including, for example, aqueous salt solutions, safflower oil, and solvents, such as ethanol, t-butanol, hexane, and glycol.

The active agent particles can be reduced in size in the presence of tyloxapol and optionally one or more secondary surface stabilizers. Alternatively, the active agent particles can be contacted with tyloxapol and optionally one or more secondary surface stabilizers after attrition. Other compounds, such as a diluent, can be added to the active agent/surface stabilizer composition during the size reduction process. Dispersions can be manufactured continuously or in a batch mode.

2. Precipitation to Obtain Nanoparticulate Drug Compositions

Another method of forming the desired nanoparticulate composition is by microprecipitation. This is a method of preparing stable dispersions of budesonide or belcomethasone in the presence of tyloxapol, optionally one or more secondary surface stabilizers, and one or more colloid stability enhancing surface active agents free of any trace toxic solvents or solubilized heavy metal impurities. Such a method comprises, for example: (1) dissolving the active agent in a suitable solvent; (2) adding the formulation from step (1) to a solution comprising tyloxapol and optionally one or more secondary surface stabilizers to form a clear solution; and (3) precipitating the formulation from step (2) using an appropriate non-solvent. The method can be followed by removal of any formed salt, if present, by dialysis or diafiltration and concentration of the dispersion by conventional means. The resultant nanoparticulate active agent dispersion can be utilized in solid or liquid dosage formulations.

3. Microfluidization to Obtain Nanoparticulate Drug Compositions

U.S. Pat. No. 5,510,118, for “Process of Preparing Therapeutic Compositions Containing Nanoparticles,” describes an exemplary method of making nanoparticulate compositions using microfluidization techniques. This patent is specifically incorporated by reference.

4. Sterile Filtration

The nanoparticulate active agent composition can be sterile filtered using conventional means. Sterile filters have pore sizes of about 0.2 microns or less, which is small enough to filter out biological contaminants. Suitable filters are commercially available.

Following sterile filtration, the nanoparticulate composition can be utilized in solid or liquid dosage formulations, such as controlled release dosage formulations, solid dose fast melt formulations, aerosol formulations, tablets, capsules, etc.

The compositions are particularly useful for dosage forms in which sterility is of primary importance, such as liquid aerosols and injectable formulations.

C. Methods of Using the Nanoparticulate Compositions

The nanoparticulate compositions of the present invention can be administered to humans and animals either orally, rectally, parenterally (intravenous, intramuscular, or subcutaneous), intracistemally, intravaginally, intraperitoneally, locally (powders, ointments or drops), or as a buccal, inhalable, or nasal spray. The beclomethasone or budesonide nanoparticulate compositions may be used in the treatment of mammals suffering from inflammatory diseases. Nanoparticulate compositions of this invention administered as inhalable aerosols are also contemplated and can be particularly useful in the treatment of respiratory illnesses, such as asthma, cystic fibrosis, chronic obstructive pulmonary disease (COPD), etc.

Pharmaceutical compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles include water, ethanol, polyols (propyleneglycol, polyethyleneglycol, glycerol, and the like), vegetable oils (such as olive oil), injectable organic esters such as ethyl oleate, and suitable mixtures thereof.

The nanoparticulate compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be achieved by the use of agents delaying absorption, such as aluminum monostearate and gelatin.

Exemplary solid dosage forms for oral administration include capsules, tablets, pills, powders, and granules.

Liquid dosage forms for oral administration include pharmaceutically acceptable emulsions, solutions, suspensions, syrups, and elixirs. In addition to the nanoparticulate compositions, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Actual dosage levels of active ingredients in the nanoparticulate compositions of the invention may be varied to obtain an amount of active ingredient that is effective to obtain a desired therapeutic response for a particular composition and method of administration. The selected dosage level therefore depends upon the desired therapeutic effect, the route of administration, the potency of the administered therapeutic compound, the desired duration of treatment, and other factors. Dosage unit compositions may contain such amounts of such submultiples thereof as may be used to make up the daily dose.

The following examples are given to illustrate the present invention. It should be understood, however, that the invention is not to be limited to the specific conditions or details described in these examples. Throughout the specification, any and all references to a publicly available document, including U.S. patents, are specifically incorporated into this patent application by reference.

### EXAMPLE 1

The purpose of this example was to prepare a sterile filtered nanoparticulate budesonide composition stabilized with tyloxapol.

Budesonide (25 g) was dispersed in an aqueous solution of tyloxapol (4.97 g) in deionized water (469.9 g). The pH of the slurry was adjusted to 4.1 with 1 M acetic acid. The slurry was process in a DYNO®-Mill (Willy A. Bachofen A G) assembled with a 300 cc chamber for continuous milling and charged with 500 μm Sdy-20 polymeric milling media (Eastman Kodak). The chamber and process fluid vessel were cooled with 10° C. coolant. Milling was performed at 4200 rpm.

After 8 hours the dispersion had a mean particle size of 161 nm and was harvested. Approximately 95 g of this dispersion was then combined with 130 cc of 50 μm SDy20 polymeric media, charged into the 150 cc batch chamber of a DYNO®-Mill, and milled at 4200 rpm. After 2 hours of milling the material was harvested and had a mean particle size of 80 nm.

The nanoparticulate budesonide dispersion was filtered through several Gelman Acrodisc PF 0.8/0.2 μm syringe filters. The mean particle size of the filtered dispersion was 83 nm, indicating that the filtration process did not significantly change the particle size distribution of the nanoparticulate budesonide dispersion.

### EXAMPLE 2

The purpose of this example was to prepare a sterile filtered nanoparticulate budesonide composition stabilized with tyloxapol and the secondary surface stabilizer hydroxypropylmethylcellulose (HPMC).

Budesonide (8.5 g) was dispersed in an aqueous solution of tyloxapol (0.85 g) and HPMC (Pharmacoat® 603; Shin-Etsu) in deionized water (74.8 g). The slurry was combined with 130 mL of 500 μm SDy20 polymeric media and charged into the 150 cc batch chamber of a DYNO®-Mill. Milling was performed at 4200 rpm. After 185 minutes the dispersion was harvested and had a mean particle size of 137 nm.

Approximately 42.5 g of this nanoparticulate budesonide dispersion was diluted with 42.5 g of deionized water and then combined with 130 mL of 50 μm SDy20 polymeric media. The material was charged into the 150 cc batch chamber of a DYNO®-Mill and milled at 4200 rpm. After 80 minutes of milling the nanoparticulate budesonide dispersion had a mean particle size of 90 nm and was harvested.

A portion of the harvested nanoparticulate budesonide dispersion was filtered through a 0.2 μm syringe filter. The mean particle size of the filtered dispersion was 87 nm, indicating that the filtration process did not significantly change the particle size distribution of the nanoparticulate budesonide dispersion.

### EXAMPLE 3

The purpose of this example was to prepare a sterile filtered nanoparticulate budesonide composition stabilized with tyloxapol using a high speed disperser.

Budesonide (210 g) was dispersed in an aqueous solution of tyloxapol (21 g) in Sterile Water for Injection, USP (819 g), and the slurry was then charged into the vessel of a Hockmeyer 5 L High Speed Disperser (Hockmeyer Equip. Corp., Harrison, N.J.). The system was placed under vacuum (20-25″ Hg) and then charged with 1365 g of 50 μm SDy20 polymeric media. Milling was performed at 7000 rpm using a centered 3″ Valynn blade. After 27 hours of milling the nanoparticulate budesonide dispersion had a mean particle size of 80 nm.

The nanoparticulate budesonide dispersion was diluted to a nominal budesonide concentration of 5% w/w and discharged. Benzalkonium chloride and acetic acid were added to the dispersion at concentrations of 0.01% w/w and 0.02%, respectively. The harvested nanoparticulate budesonide dispersion was filtered through a Gelman SuporCap 0.8/0.2 μm sterilizing grade capsule filter and assayed for budesonide concentration which was found to be 5.0% w/w.

### EXAMPLE 4

The purpose of this example was to prepare a sterile filtered nanoparticulate budesonide composition stabilized with tyloxapol and the secondary surface stabilizer polyvinylpyrrolidone using a high speed disperser.

Budesonide (210 g) was dispersed in an aqueous solution of tyloxapol (21 g) and polyvinylpyrrolidone (21 g) in Sterile Water for Injection, USP (798 g), and the slurry was then charged into the vessel of a Hockmeyer 5 L High Speed Disperser. The system was placed under vacuum (20-25″ Hg) and then charged with 1365 g of 50 μm SDy20 polymeric media. Milling was performed at 7000 rpm using a centered 3″ Valynn blade. After 27 hours of milling the nanoparticulate budesonide dispersion had a mean particle size of 80 nm.

The nanoparticulate budesonide dispersion was diluted to a nominal budesonide concentration of 5% w/w and discharged. Benzalkonium chloride and acetic acid were added to the dispersion at concentrations of 0.01% w/w and 0.02%, respectively. The harvested nanoparticulate budesonide dispersion was filtered through a Gelman SuporCap 0.8/0.2 μm sterilizing grade capsule filter and assayed for budesonide concentration which was found to be 5.0% w/w.

### EXAMPLE 5

The purpose of this example was to demonstrate the inability to sterile filter a nanoparticulate composition of budesonide stabilized with hydroxypropyl methylcellulose.

Budesonide (8.54 g) was dispersed in an aqueous solution of hydroxypropylmethyl cellulose (Methocel E3 Premium LV; Dow Chemical) (1.72 g) in deionized water (74.83 g). Approximately 75 g of the slurry was combined with 130 cc of 500 μm SDy20 polymeric media and charged into the 150 cc batch chamber of a DYNO®-Mill. Milling was performed at 4200 rpm. After 4 hours the dispersion had a mean particle size of 128 nm.

The nanoparticulate budesonide dispersion was harvested and diluted with water to yield 79 g of a dispersion with a nominal budesonide concentration of 5% w/w.

Approximately 75 g of this nanoparticulate budesonide dispersion was then combined with 140 mL of 50 μm SDy20 polymeric media. An additional 10 mL of deionized water was added to reduce the viscosity of the dispersion. The material was charged into the 150 cc batch chamber of a DYNO®-Mill and milled at 4200 rpm. After 4 hours of milling the material was harvested and diluted with an additional 36 111L of water. The resulting nanoparticulate budesonide dispersion had a mean particle size of 89 nm but was somewhat aggregated.

An attempt was made to filter a small aliquot through a 25 mm Gelman Supor 0.8/0.2 polyethersulfone syringe filter, however the effluent was clear indicating that the therapeutic compound particles were unable to pass through the filter pores.

### EXAMPLE 6

The purpose of this example was to demonstrate the inability to sterile filter a nanoparticulate budesonide composition stabilized with methyl cellulose.

Budesonide (8.5 g) was dispersed in an aqueous solution of methyl cellulose (Methocel A15 Premium LV; Dow Chemical) (1.72 g) in deionized water (74.94 g). The slurry was combined with 130 mL of 500 μm SDy20 polymeric media and charged into the 150 cc batch chamber of a DYNO®-Mill. Milling was performed at 4200 rpm. After 4 hours the nanoparticulate budesonide dispersion was harvested and diluted with ca. 30 mL of water to yield a nominal budesonide concentration of 5% w/w.

The nanoparticulate budesonide dispersion had a mean particle size of 170 nm. Approximately 60 g of this dispersion was then combined with 120 mL of 50 μm SDy20 polymeric media. An additional 10 μL of deionized water was added to increase the volume of the dispersion. The material was charged into the 150 cc batch chamber of a DYNO®-Mill and milled at 4200 rpm. After 2 hours of milling the material was harvested and diluted with an additional 40 mL of water to reduce the viscosity.

The resulting nanoparticulate budesonide dispersion consisted of aggregated budesonide particles having an average size of approximately 2 microns, and was therefore unsuitable for 0.2 μm sterile filtration.

### EXAMPLE 7

The purpose of this example was to demonstrate the inability to sterile filter a nanoparticulate budesonide composition stabilized with Pluronic® F108.

Budesonide (8.51 g) was dispersed in an aqueous solution of Pluronic® F108 (1.69 g) in deionized water (74.84 g). The slurry was combined with 130 cc of 500 μm SDy20 polymeric media and charged into the 150 cc batch chamber of a DYNO®-Mill. Milling was performed at 4200 rpm. After 4 hours the nanoparticulate budesonide dispersion had a mean particle size of 276 nm.

The nanoparticulate budesonide dispersion was immediately harvested and found to have a mean particle size of 739 nm, indicating that very rapid crystal growth had occurred. Due to this instability, the nanoparticulate budesonide dispersion was deemed unsuitable for 0.2 μm sterile filtration.

### EXAMPLE 8

The purpose of this example was to demonstrate the inability to sterile filter a nanoparticulate budesonide composition stabilized with polysorbate 80.

Budesonide (8.5 g) was dispersed in a solution of polysorbate 80 (1.7 g) in aqueous diluent (74.8 g). The slurry was combined with 130 mL of 500 μm SDy20 polymeric media and charged into the 150 cc batch chamber of a DYNO®-Mill. Milling was performed at 4200 rpm. After 2 hours the nanoparticulate budesonide dispersion was harvested and had a mean particle size of 221 nm.

Approximately 42.5 g of this dispersion was diluted with 42.5 g of aqueous diluent and then combined with 120 mL of 50 μm SDy20 polymeric media. The material was charged into the 150 cc batch chamber of a DYNO®-Mill and milled at 4200 rpm. After 1 hour of milling the nanoparticulate budesonide dispersion had a mean particle size of 216 nm, and after 2 hours the average size had decreased to only 192 nm, indicating that no significant additional particle size reduction had taken place.

Because of the large average particle size of the nanoparticulate budesonide dispersion, the material was unsuitable for 0.2 μm sterile filtration.

### EXAMPLE 9

The purpose of this example was to demonstrate the inability to sterile filter a nanoparticulate budesonide composition stabilized with polysorbate 80 and polyvinylpyrrolidone.

Budesonide (8.5 g) was dispersed in an aqueous solution of polysorbate 80 (0.85 g) and polyvinylpyrrolidone (0.85 g) in deionized water (74.8 g). The slurry was combined with 130 mL of 500 μm SDy20 polymeric media and charged into the 150 cc batch chamber of a DYNO®-Mill. Milling was performed at 4200 rpm. After 180 minutes the nanoparticulate budesonide dispersion was harvested and had a mean particle size of 232 nm.

Approximately 40 g of this nanoparticulate budesonide dispersion was diluted with 40 g of deionized water and then combined with 120 mL of 50 μm SDy20 polymeric media. The material was charged into the 150 cc batch chamber of a DYNO®-Mill and milled at 4200 rpm. After 180 minutes of milling the nanoparticulate budesonide dispersion was harvested and had a mean particle size of 203 nm.

Because of the large average particle size of the nanoparticulate budesonide dispersion, the material was unsuitable for 0.2 μm sterile filtration.

### EXAMPLE 10

The purpose of this example was prepare a sterile filtered nanoparticulate beclomethasone composition stabilized with tyloxapol and the secondary surface stabilizer polyvinylpyrrolidone.

Beclomethasone dipropionate (4.25 g) was dispersed in an aqueous solution of tyloxapol (0.85 g) and polyvinylpyrrolidone (0.85 g) in deionized water (79.05 g). The slurry was combined with 120 cc of 50 μm SDy20 polymeric media and charged into the 150 cc batch chamber of a DYNO®-Mill. Milling was performed at 4200 rpm. After 1.5 hours the dispersion had a mean particle size of 97 nm.

A portion of the nanoparticulate beclomethasone dispersion was filtered through a 0.2 μm syringe sterile filter.

### EXAMPLE 11

The purpose of this example was prepare a sterile filtered nanoparticulate beclomethasone composition stabilized with tyloxapol.

Beclomethasone dipropionate (4.25 g) was dispersed in an aqueous solution of tyloxapol (0.85 g) in deionized water (79.9 g). The slurry was combined with 120 cc of 50 μm SDy20 polymeric media and charged into the 150 cc batch chamber of a DYNO®-Mill. Milling was performed at 4200 rpm. After 1.5 hours the nanoparticulate beclomethasone dispersion had a mean particle size of 98 nm.

The nanoparticulate beclomethasone dispersion was harvested and a portion of the material was filtered through a 0.2 μm syringe filter. The mean particle size of the filtered nanoparticulate beclomethasone dispersion was 97 nm, indicating that no significant change to the particle size distribution had occurred as a result of sterile filtration.

### EXAMPLE 12

The purpose of this example was to demonstrate the inability to sterile filter a nanoparticulate beclomethasone composition stabilized with polysorbate 80.

Beclomethasone dipropionate (4.50 g) was dispersed in an aqueous solution of polysorbate 20 (0.90 g) in deionized water (84.6 g). The slurry was combined with 130 mL of 500 μm SDy20 polymeric media and charged into the 150 cc batch chamber of a DYNO®-Mill. Milling was performed at 4200 rpm. After 125 minutes the dispersion had a mean particle size of 241 nm.

The nanoparticulate beclomethasone dispersion was immediately harvested and the mean particle size was found to have increased to 375 nm, indicating that very rapid crystal growth had occurred. Due to this instability, the nanoparticulate beclomethasone dispersion was deemed unsuitable for 0.2 μm sterile filtration.

### EXAMPLE 13

The purpose of this example was to demonstrate the inability to sterile filter a nanoparticulate beclomethasone composition stabilized with polysorbate 20.

Beclomethasone dipropionate (4.50 g) was dispersed in an aqueous solution of polysorbate 20 (0.90 g) in deionized water (84.61 g). The slurry was combined with 130 mL of 500 μm SDy20 polymeric media and charged into the 150 cc batch chamber of a DYNO®-Mill. Milling was performed at 4200 rpm. After 1 hour the dispersion had a mean particle size of 212 nm, and after 2 hours the average size had decreased to only 193 nm indicating that no significant additional particle size reduction had taken place. Furthermore, the dispersion was significantly aggregated.

Because of the large average particle size of the nanoparticulate beclomethasone dispersion and its degree of aggregation, the material was unsuitable for 0.2 μm sterile filtration.

### EXAMPLE 14

The purpose of this example was to demonstrate the inability to sterile filter a nanoparticulate beclomethasone composition stabilized with polyvinylpyrrolidone.

Beclomethasone dipropionate (4.5 g) was dispersed in an aqueous solution of polyvinylpyrrolidone (0.90 g) in deionized water (84.6 g). The slurry was combined with 130 mL of 500 μm SDy20 polymeric media and charged into the 150 cc batch chamber of a DYNO®-Mill. Milling was performed at 4200 rpm. After 1 hour the dispersion had a mean particle size of 389 nm, and after two hours the mean particle size was 387 nm, indicating that no further size reduction had taken place. The dispersion was also highly aggregated.

Due to the large particle size and extent of aggregation the nanoparticulate beclomethasone dispersion was deemed unsuitable for 0.2 μm sterile filtration.

### EXAMPLE 15

The purpose of this example was to demonstrate the inability to sterile filter a nanoparticulate flunisolide composition stabilized with tyloxapol.

Flunisolide is an anti-inflammatory steroid having the chemical name 6α-fluoro-11β, 16α, 17,21-tetrahydroxy-pregna-1,4-diene-3,20-dione cyclic-16,17-acetal with acetone. It is practically insoluble in water.

Flunisolide (8.5 g) was dispersed in an aqueous solution of tyloxapol (1.7 g) and sodium chloride (1.53 g) in deionized water (73.27 g). The slurry was combined with 130 mL of 500 μm SDy20 polymeric media and charged into the 150 cc batch chamber of a DYNO®-Mill. Milling was performed at 4200 rpm. After 1.5 hours the nanoparticulate flunisolide dispersion was harvested and had a mean particle size of 115 nm.

Approximately 42.5 g of this nanoparticulate flunisolide dispersion was diluted with 42.5 g of deionized water and then combined with 120 mL of 50 μm SDy20 polymeric media. The material was charged into the 150 cc batch chamber of a DYNO®-Mill and milled at 4200 rpm. After 2 hours of milling the nanoparticulate flunisolide dispersion was harvested and had a mean particle size of 99 nm.

In spite of the relatively small particle size of the nanoparticulate flunisolide dispersion, the material could not be filtered through Gelman Supor 0.45 μm or 0.8/0.2 μm polyethersulfone syringe filters.

### EXAMPLE 16

The purpose of this example was to demonstrate the inability to sterile filter a nanoparticulate triamcinolone acetonide composition stabilized with tyloxapol.

Triamcinolone acetonide is a corticosteroid with the chemical designation 9-Fluoro-11b, 16a, 17,21-tetrahydroxypregna-1,4-diene-3,20-dione cyclic 16,17-acetal with acetone (C₂₄H₃₁FO₆).

Triamcinolone acetonide (4.25 g) was dispersed in an aqueous solution of tyloxapol (0.85 g) in deionized water (79.90 g). The slurry was combined with 130 cc of 500 μm SDy20 polymeric media and charged into the 150 cc batch chamber of a DYNO®-Mill. Milling was performed at 4200 rpm. After 1 hour the dispersion had a mean primary particle size of 164 nm but was highly aggregated, and after two hours the mean primary particle size was 157 nm, indicating that no significant additional size reduction had taken place.

The nanoparticulate triamcinolone acetonide dispersion remained highly aggregated with the average aggregate size being approximately 3 μm. Due to the large particle size and extent of aggregation the nanoparticulate triamcinolone acetonide dispersion was deemed unsuitable for 0.2 μm sterile filtration.

### EXAMPLE 17

The purpose of this example was to demonstrate the inability to sterile filter a nanoparticulate triamcinolone acetonide composition stabilized with tyloxapol.

Triamcinolone acetonide (4.25 g) was dispersed in an aqueous solution of tyloxapol (2.13 g) in deionized water (78.62 g). The slurry was combined with 130 cc of 500 μm SDy20 polymeric media and charged into the 150 cc batch chamber of a DYNO®-Mill. Milling was performed at 4200 rpm. After 1 hour the dispersion had a mean primary particle size of 171 nm but was highly aggregated, and after two hours the mean primary particle size was 144 nm, indicating that very little additional size reduction had taken place.

The nanoparticulate triamcinolone acetonide dispersion remained highly aggregated with the average aggregate size being approximately 3.7 μm. Due to the large particle size and extent of aggregation the nanoparticulate triamcinolone acetonide dispersion was deemed unsuitable for 0.2 μm sterile filtration.

### EXAMPLE 18

The purpose of this example was to demonstrate the inability to sterile filter a nanoparticulate triamcinolone acetonide composition stabilized with tyloxapol and the secondary surface stabilizer polyvinylpyrrolidone.

Triamcinolone acetonide (4.25 g) was dispersed in an aqueous solution of tyloxapol (0.85 g) and poylvinylpyrrolidone (0.85 g) in deionized water (79.05 g). The slurry was combined with 130 cc of 500 μm SDy20 polymeric media and charged into the 150 cc batch chamber of a DYNO®-Mill. Milling was performed at 4200 rpm. After 1 hour the dispersion had a mean primary particle size of 152 nm but was highly aggregated, and after two hours the mean primary particle size was 117 nm, indicating that relatively little additional size reduction had taken place.

The nanoparticulate triamcinolone acetonide dispersion remained highly aggregated with the average aggregate size being approximately 1 μm. Due to the large particle size and extent of aggregation the nanoparticulate triamcinolone acetonide dispersion was deemed unsuitable for 0.2 μm sterile filtration.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. A nanoparticulate composition comprising:
(a) nanoparticulate beclomethasone particles, budesonide particles, or a combination thereof, having an effective average particle size of less than about 150 nm; and
(b) tyloxapol as a surface stabilizer adsorbed onto the surface of said beclomethasone and/or budesonide particles,
wherein the nanoparticulate composition is sterile filtered.

2. The composition of claim 1, wherein the beclomethasone particles, budesonide particles, or a combination thereof are present in an amount selected from the group consisting of about 99% to about 1% (w/w), about 90% to about 10% (w/w), about 80% to about 30%, and about 80% to about 40% (w/w), based on the total combined dry weight of beclomethasone, budesonide, and tyloxapol.

3. The composition of claim 1, wherein the concentration of tyloxapol is selected from the group consisting of from about 0.01 to about 90%, from about 1 to about 75%, from about 10 to about 60%, and from about 10 to about 30% by weight, based on the total combined dry weight of beclomethasone, budesonide, and tyloxapol.

4. The composition of claim 1, wherein the effective average particle size of the beclomethasone particles, budesonide particles, or a combination thereof is less than about 120 nm.

5. The composition of claim 1 wherein the effective average particle size of the beclomethasone particles, budesonide particles, or a combination thereof is less than about 100 nm.

6. The composition of claim 1 wherein the effective average particle size of the beclomethasone particles, budesonide particles, or a combination thereof is less than about 80 nm.

7. The composition of claim 1 wherein the effective average particle size of the beclomethasone particles, budesonide particles, or a combination thereof is less than about 50 nm.

8. The composition of claim 1 further comprising at least one secondary surface stabilizer.

9. The composition of claim 8, wherein the secondary surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, poloxamers, poloxamines, charged phospholipids, dioctylsulfosuccinate, Tetronic 1508®, dialkylesters of sodium sulfosuccinic acid, sodium lauryl sulfates, alkyl aryl polyether sulfonates, mixtures of sucrose stearate and sucrose distearate, p-isononylphenoxypoly-(glycidol), C₁₈H₃₇CH₂(CON(CH₃)—CH₂(CHOH)₄(CH₂OH)₂, decanoyl-N-methylglucamide, n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, and random copolymers of vinyl acetate and vinyl pyrrolidone.

10. The composition of claim 8, wherein the secondary surface stabilizer is selected from the group consisting of dioctylsulfosuccinate, sodium lauryl sulfate, hydroxypropylmethyl cellulose, benzalkonium chloride, and polyvinylpyrrolidine.

11. The composition of claim 1, wherein the beclomethasone and/or budesonide particles are crystalline, semi-crystalline, amorphous, semi-amorphous, or a mixture thereof.

12. The composition of claim 1, further comprising one or more pharmaceutically acceptable excipients.

13. The composition of claim 1, wherein the beclomethasone is beclomethasone dipropionate.

14. The composition of claim 1 formulated into an aerosol for nasal or pulmonary administration.

15. A method of making a nanoparticulate composition comprising:
(a) dispersing particles of budesonide, beclomethasone, or a mixture thereof in a liquid dispersion medium; and
(b) applying mechanical means in the presence of grinding media to reduce the average particle size of budesonide, beclomethasone, or a mixture thereof in the liquid dispersion medium to less than about 150 nm, and
(c) sterile filtering the resulting nanoparticulate dispersion;
wherein tyloxapol is added to the liquid dispersion medium before or after milling.

16. The method of claim 15, wherein the beclomethasone particles, budesonide particles, or a combination thereof are present in an amount selected from the group consisting of about 99% to about 1% (w/w), about 90% to about 10% (w/w), about 80% to about 30%, and about 80% to about 40% (w/w), based on the total combined dry weight of beclomethasone, budesonide, and tyloxapol.

17. The method of claim 15, wherein the concentration of tyloxapol is selected from the group consisting of from about 0.01 to about 90%, from about 1 to about 75%, from about 10 to about 60%, and from about 10 to about 30% by weight, based on the total combined dry weight of beclomethasone, budesonide, and tyloxapol.

18. The method of claim 15, wherein the effective average particle size of the beclomethasone particles, budesonide particles, or a combination thereof is selected from the group consisting of less than about 120 nm, less than about 100 nm, less than about 80 nm, and less than about 50 nm.

19. The method of claim 15 further comprising adding at least one secondary surface stabilizer to the liquid dispersion medium before or after milling.

20. The method of claim 19, wherein the secondary surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, poloxamers, poloxamines, charged phospholipids, dioctylsulfosuccinate, Tetronic 1508®, dialkylesters of sodium sulfosuccinic acid, sodium lauryl sulfates, alkyl aryl polyether sulfonates, mixtures of sucrose stearate and sucrose distearate, p-isononylphenoxypoly-(glycidol), C₁₈H₃₇CH₂(CON(CH₃)—CH₂(CHOH)₄(CH₂OH)₂, decanoyl-N-methylglucamide, n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, and random copolymers of vinyl acetate and vinyl pyrrolidone.

21. The method of claim 19, wherein the secondary surface stabilizer is selected from the group consisting of dioctylsulfosuccinate, sodium lauryl sulfate, hydroxypropylmethyl cellulose, benzalkonium chloride, and polyvinylpyrrolidine.

22. The method of claim 15, wherein the beclomethasone and/or budesonide particles are crystalline, semi-crystalline, amorphous, semi-amorphous, or a mixture thereof.

23. A method of making a nanoparticulate composition comprising:
(a) dissolving beclomethasone, budesonide, or a combination thereof in a solvent;
(b) adding the solubilized beclomethasone, budesonide, or a combination thereof to a solution comprising tyloxapol to form a clear solution;
(c) precipitating the solubilized beclomethasone, budesonide, or a combination thereof having tyloxapol adsorbed on the surface thereof using a non-solvent; and
(d) sterile filtering the resulting nanoparticulate dispersion,
wherein the resulting composition of nanoparticulate beclomethasone, budesonide, or a combination thereof has an effective average particle size of less than about 150 nm.

24. The method of claim 23, wherein the beclomethasone particles, budesonide particles, or a combination thereof are present in an amount selected from the group consisting of about 99% to about 1% (w/w), about 90% to about 10% (w/w), about 80% to about 30%, and about 80% to about 40% (w/w), based on the total combined dry weight of beclomethasone, budesonide, and tyloxapol.

25. The method of claim 23, wherein the concentration of tyloxapol is selected from the group consisting of from about 0.01 to about 90%, from about 1 to about 75%, from about 10 to about 60%, and from about 10 to about 30% by weight, based on the total combined dry weight of beclomethasone, budesonide, and tyloxapol.

26. The method of claim 23, wherein the effective average particle size of the beclomethasone particles, budesonide particles, or a combination thereof is selected from the group consisting of less than about 120 nm, less than about 100 nm, less than about 80 nm, and less than about 50 nm.

27. The method of claim 23 further comprising adding at least one secondary surface stabilizer to the liquid dispersion medium before or after milling.

28. The method of claim 27, wherein the secondary surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, poloxamers, poloxamines, charged phospholipids, dioctylsulfosuccinate, Tetronic 1508®, dialkylesters of sodium sulfosuccinic acid, sodium lauryl sulfates, alkyl aryl polyether sulfonates, mixtures of sucrose stearate and sucrose distearate, p-isononylphenoxypoly-(glycidol), C₁₈H₃₇CH₂(CON(CH₃)—CH₂(CHOH)₄(CH₂OH)₂, decanoyl-N-methylglucamide, n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, and random copolymers of vinyl acetate and vinyl pyrrolidone.

29. The method of claim 27, wherein the secondary surface stabilizer is selected from the group consisting of dioctylsulfosuccinate, sodium lauryl sulfate, hydroxypropylmethyl cellulose, benzalkonium chloride, and polyvinylpyrrolidine.

30. The method of claim 23, wherein the beclomethasone and/or budesonide particles are crystalline, semi-crystalline, amorphous, semi-amorphous, or a mixture thereof.

31. A method of treating a patient in need with a nanoparticulate composition comprising administering to a patient in need a therapeutically effective amount of a nanoparticulate composition of budesonide, beclomethasone, or a combination thereof, wherein said composition comprises:
(a) budesonide, beclomethasone, or a combination thereof having an effective average particle size of less than about 150 nm; and
(b) tyloxapol adsorbed on the surface of the budesonide and/or beclomethasone,
wherein the nanoparticulate composition has been sterile filtered.

32. The method of claim 31, wherein said treatment is for an inflammatory disease.

33. The method of claim 31, wherein said treatment is for asthma, cystic fibrosis, or chronic obstructive pulmonary disease.

34. The method of claim 31, wherein said composition is administered via a nasal or pulmonary aerosol.
